(19) United States
(12) Patent Application Publication
(10) Pub. No.: US 2026/0055432 A1
(43) Pub. Date: Feb. 26, 2026

(54) NANOBUBBLES FOR FERMENTATION PROCESSES

(71) Applicant: Moleaer, Inc., Hawthorne, CA (US)

(72) Inventors: Thuy Thi Bui, Torrance, CA (US); Sohail Akhter, Carson, CA (US)

(21) Appl. No.: 19/306,418

(22) Filed: Aug. 21, 2025

Related U.S. Application Data
(60) Provisional application No. 63/686,265, filed on Aug. 23, 2024.

(51) Int. Cl. C12P 7/06

(52) U.S. Cl. CPC C12P 7/06

(57) ABSTRACT

Provided herein are fermentation processes that involve the use of nanobubbles. One such process includes providing a fermentable composition comprising (i) a sterilized nanobubble composition comprising nanobubbles in a liquid carrier, (ii) a feedstock, and (iii) a microorganism; fermenting the fermentable composition to produce a fermentation product and fermentation residue; and separating the fermentation product from the fermentation residue.

### CROSS-REFERENCE TO RELATED APPLICATION

This application claims the benefit of U.S. Provisional Application No. 63/686,265, filed Aug. 23, 2024, the disclosure of which is incorporated herein by reference in its entirety.

### TECHNICAL FIELD

This disclosure relates to fermentation processes.

### BACKGROUND

Fermentation is one of the oldest and most widely used biotechnology processes, with applications ranging from food and beverage production to pharmaceutical manufacturing. Fermentation involves the metabolic breakdown of organic substances by microorganisms to produce desired compounds. Fermentation has been practiced for thousands of years, with early uses focused on food preservation and flavor enhancement. In recent decades, advances in biotechnology have expanded the applications of fermentation to produce a wide range of valuable products, including antibiotics, enzymes, and biofuels.

### SUMMARY

The inventors have discovered that nanobubbles can survive sterilization processes, thus enabling nanobubbles to be used in large-scale fermentation processes that require sterilization. Specifically, a fermentable composition including a sterilized nanobubble composition comprising nanobubbles in a liquid carrier, a feedstock, and a microorganism is fermented in a fermentation tank. In some examples, the fermentable composition is formed by sterilizing a nanobubble composition and a feedstock in a fermentation tank. In some examples, a nanobubble composition including nanobubbles in a liquid carrier is sterilized upstream of a fermentation tank and the sterilized nanobubble composition is added to the tank for fermentation.

As used herein, a “sterilization process” is a process that uses moist heat, dry heat, pressure, irradiation, chemicals, electromagnetic fields, sonication, or combinations thereof to reduce microbial and/or viral contaminants that could interfere with the fermentation process. A “sterilized composition” is a composition that has been subjected to a sterilization process. A “sterilizable composition” is a composition that is capable of being subjected to a sterilization process.

Accordingly, aspects of the present disclosure provide a fermentation process comprising (a) providing, in a fermentation tank, a fermentable composition comprising (i) a sterilized nanobubble composition comprising nanobubbles in a liquid carrier, (ii) a feedstock, and (iii) a microorganism; (b) fermenting the fermentable composition to produce a fermentation product and fermentation residue; and (c) separating the fermentation product from the fermentation residue. See FIG. 1.

In some embodiments, fermentation processes comprise (a) adding (i) a nanobubble composition comprising nanobubbles in a liquid carrier and (ii) feedstock to the fermentation tank; (b) sterilizing the contents of the tank; and (c) adding the microorganism to the tank.

In some embodiments, fermentation processes comprise (a) combining a sterilized precursor composition comprising nanobubbles in a liquid carrier with a feedstock to form a precursor composition; (b) adding the precursor composition to the fermentation tank; and (c) adding the microorganism to the tank.

In some embodiments, fermentation processes comprise (a) combining a sterilized composition comprising nanobubbles in a liquid carrier with a feedstock to form a precursor composition; and (b) combining the precursor composition with a microorganism present in the fermentation tank.

In some embodiments, fermentation processes comprise (a) forming a sterilized precursor composition comprising nanobubbles in a liquid carrier and a feedstock; (b) adding the sterilized precursor composition to the fermentation tank; and (c) adding the microorganism to the tank.

In some embodiments, fermentation processes comprise (a) forming a sterilized precursor composition comprising nanobubbles in a liquid carrier and a feedstock; and (b) combining the sterilized precursor composition with a microorganism present in the fermentation tank.

In some embodiments, forming the sterilized precursor composition comprises combining a sterilized nanobubble composition comprising nanobubbles in a liquid carrier with a sterilized feedstock to form the sterilized precursor composition.

In some embodiments, forming the sterilized precursor composition comprises (a) combining a nanobubble composition comprising nanobubbles in a liquid carrier with a feedstock to form a sterilizable composition; and (b) sterilizing the sterilizable composition to form the sterilized precursor composition.

In some embodiments, fermentation processes comprise forming the precursor composition upstream of and in-line with the fermentation tank.

In some embodiments, fermentation processes comprise forming the sterilized precursor composition upstream of and in-line with the fermentation tank.

In some embodiments, the concentration of nanobubbles in the nanobubble composition comprising nanobubbles in a liquid carrier is at least 10⁶ nanobubbles per cm³, at least 10⁷ nanobubbles per cm³, at least 10⁸ nanobubbles per cm³, at least 10⁹ nanobubbles per cm³, at least 10¹⁰ nanobubbles per cm³, or at least 10¹¹ nanobubbles per cm³.

In some embodiments, fermentation processes comprise conducting the fermentation process using a continuous introduction of the nanobubble composition comprising nanobubbles in a liquid carrier.

In some embodiments, fermentation processes comprise conducting the fermentation process using a single introduction of the nanobubble composition comprising nanobubbles in a liquid carrier.

In some embodiments, the liquid carrier comprises an aqueous carrier.

In some embodiments, the microorganism is selected from the group consisting of bacteria, fungi, yeast, and combinations thereof.

Other features and advantages of the disclosure will be apparent from the following detailed description, and from the claims.

### BRIEF DESCRIPTION OF DRAWINGS

FIG. 1 is a flow chart of a fermentation process involving use of nanobubbles.

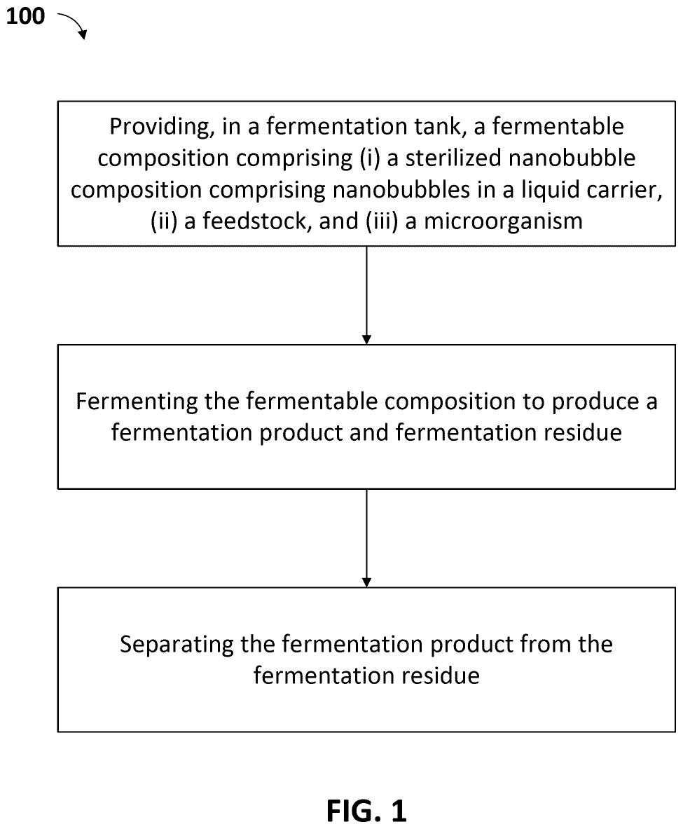

FIG. 2 is a schematic depiction of an example of a submerged batch fermentation (SBF) process involving use of nanobubbles.

FIG. 3 is a schematic depiction of an example of a submerged fed-batch fermentation (SFF) process involving use of nanobubbles.

FIG. 4 is a schematic depiction of an example of a submerged continuous fermentation (SCF) process involving use of nanobubbles.

FIG. 5 is a schematic depiction of an example of a solid state fermentation (SSF) process involving use of nanobubbles.

### DETAILED DESCRIPTION

Following below are more detailed descriptions of various concepts related to, and exemplary embodiments of, inventive processes related to using sterilized compositions including nanobubbles in fermentation. Aspects of the present disclosure provide large-scale fermentation processes in which nanobubbles survive sterilization processes to enhance overall fermentation process efficiency.

Any type of fermentation known in the art or described herein can be used in fermentation processes described herein. Non-limiting examples of types of fermentation include batch fermentation, continuous fermentation, fed-batch fermentation, anaerobic fermentation, aerobic fermentation, surface fermentation, submerged fermentation, solid state fermentation, submerged batch fermentation, submerged fed-batch fermentation, submerged continuous fermentation, solid state fermentation, and combinations of any of these.

Any of the fermentation processes described herein includes use of a nanobubble composition comprising nanobubbles in a liquid carrier. Alternatively, or in addition to, any of the fermentation processes described herein can include generating nanobubbles in a liquid carrier. Generally, because nanobubbles can survive sterilization processes, nanobubble-containing liquid is sterilized in conjunction with the fermentation processes described here. In some examples, nanobubble-containing liquid is sterilized, e.g., in a batch or continuous flow process, prior to pumping the nanobubble-containing liquid into a fermentation tank. In some examples, nanobubble-containing liquid is sterilized in-situ in the fermentation tank in a batch sterilization process.

As used herein, the term “nanobubble” refers to a bubble that has a diameter of less than one micron. A microbubble, which is larger than a nanobubble, is a bubble that has a diameter greater than or equal to one micron and smaller than 50 microns. A macrobubble is a bubble that has a diameter greater than or equal to 50 microns. As used herein, a “nanobubble generator” refers to a device for generating nanobubbles.

Nanobubbles have several unique properties such as high surface area to volume ratio, long-term stability in solution, and neutral buoyancy, which can enhance gas-liquid mass transfer efficiency. These properties make nanobubbles advantageous for improving oxygen transfer and enhancing microbial activity in aerobic fermentation processes, e.g., contributing to improvements in fermentation efficiency. Moreover, nanobubbles can boost cell doubling rates, increase peak cell biomass, and reduce fermentation time, which can result in higher yields and/or lower costs for fermentation processes.

Any suitable concentration of nanobubbles can be included in a nanobubble composition comprising nanobubbles in a liquid carrier for use in methods described herein. In some embodiments, the nanobubble composition includes nanobubbles at a concentration of at least 10⁶ nanobubbles per cm³, at least 10⁷ nanobubbles per cm³, at least 10⁸ nanobubbles per cm³, at least 10⁹ nanobubbles per cm³, at least 10¹⁰ nanobubbles per cm³, at least 10¹¹ nanobubbles per cm³, or more. In some embodiments, the concentration of nanobubbles can be at saturation in the nanobubble composition.

Nanobubbles for use in methods described herein can include any suitable gas. Non-limiting examples of gases include air, hydrogen, biogas, methane, carbon dioxide, nitrogen, or oxygen.

Nanobubbles for use in methods described herein have a diameter of less than one micron, e.g., between 10-500 nanometers, between 50-300 nanometers, or between 50-200 nanometers. The nanobubbles can have a zeta potential between −5 to −30 mV in typical clean or tap water.

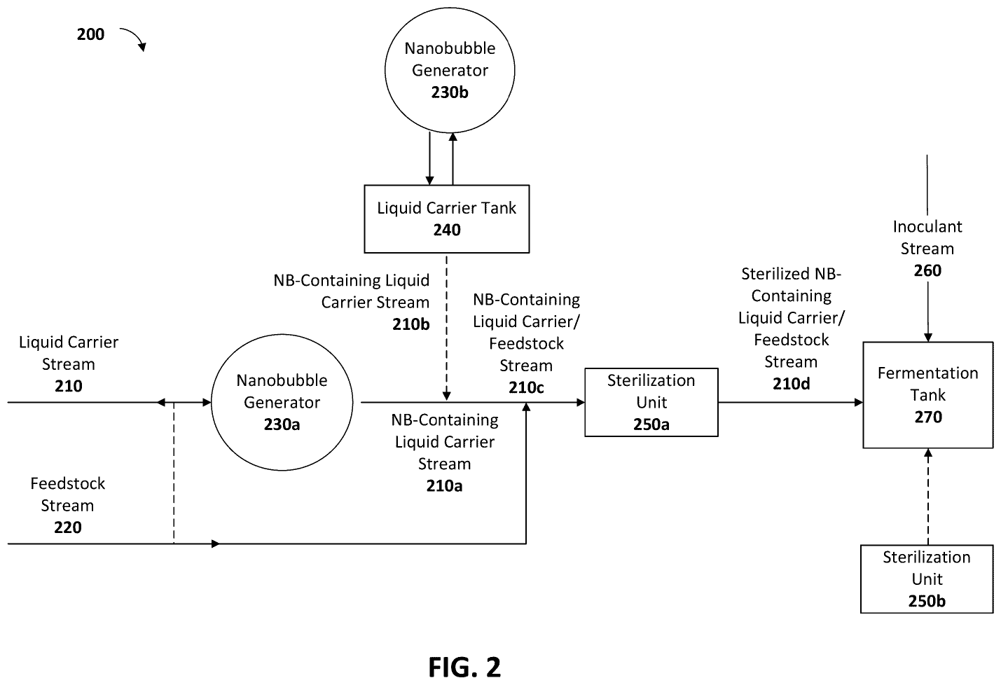

FIGS. 2-5 are examples of fermentation processes that involve sterilizable compositions containing nanobubbles.

Referring to FIG. 2, an example of a submerged batch fermentation process 200 using nanobubbles is described.

A liquid carrier stream 210 (e.g., a water stream) is flowed into a nanobubble generator 230a where nanobubbles are generated in liquid carrier stream 210 to produce a nanobubble-containing liquid carrier stream 210a. In some implementations, the liquid carrier stream 210 contains only liquid carrier, and in some implementations, the liquid carrier stream 210 contains both liquid carrier and feedstock.

The nanobubble generator 230a (and other nanobubble generators described herein) can implement any suitable method known in the art or described herein to generate nanobubbles in a liquid carrier. Non-limiting examples of methods and apparatuses for generating nanobubbles that can be used in methods described herein are provided in U.S. patent application Ser. Nos. 10,591,231, and 11,331,633, the entire contents of which are herein incorporated by reference for the purposes and subject matter referenced herein.

Fermentation processes described herein can include more than one introduction of nanobubble-containing liquid carrier. For example, as shown in FIG. 2, a nanobubble-containing liquid carrier stream 210b from a liquid carrier tank 240 can be mixed with the nanobubble-containing liquid carrier stream 210a. Nanobubbles are generated in liquid carrier from the liquid carrier tank 240 by generating nanobubbles in the liquid carrier along a recirculating flow path using a nanobubble generator 230b.

Feedstock can be mixed with liquid carrier before or after nanobubbles are generated in the liquid carrier. Feedstock can include nutrients and/or media for fermentation of microorganisms. In some embodiments, the feedstock can include a carbon source, a nitrogen source, or combinations thereof. In some embodiments, the carbon source includes simple carbon-containing substrates, complex carbon-containing substrates, or combinations thereof. For example, the simple carbon-containing substrate can include, but is not limited to, glucose (e.g., dextrose), glycerol, ethanol, acetate, methanol, or combination thereof. In some embodiments, the complex carbon-containing substrate can include, but is not limited to, sucrose, sugar thick juice, molasses, starch, hydrolysate, lactose, whey, or combinations thereof.

In some embodiments, glucose (e.g., dextrose) can be used as a feedstock during fermentation to produce industrial products that include amino acids, antibiotics, enzymes, ethanol, or combinations thereof. In some embodiments, sucrose can be used during fermentation to produce industrial products that include yeast, citric acids, vitamins, or combinations thereof. In some embodiments, sugar thick juice can be used during fermentation to produce industrial products that include organic acids, polysaccharides, or combinations thereof. In some embodiments, molasses can be used during fermentation to produce industrial products that include fuel ethanol, baker's yeast, feed yeast, or combinations thereof. In some embodiments, starch and/or hydrolysate can be used during fermentation to produce industrial products that include lactic acid, probiotics, pharmaceutical proteins, or combinations thereof. In some embodiments, lactose and/or whey can be used during fermentation to produce industrial products that include probiotic cultures, β-galactosidase, or combinations thereof. In some embodiments, glycerol can be used during fermentation to produce industrial products that include 1,3-propanediol, polyhydroxybutyrate, docosahexaenoic acid, or combinations thereof. In some embodiments, ethanol can be used during fermentation to produce industrial products that include polyhydroxybutyrate, 3-hydroxypropionic acid, acetyl coenzyme-A chemicals, or combinations thereof. In some embodiments, acetate can be used during fermentation to produce industrial products that include lipids, terpenoids, polyketides, or combinations thereof. In some embodiments, methanol can be used during fermentation to produce industrial products such as recombinant proteins, amino acids, or combinations thereof.

In some embodiments, the nitrogen source includes simple nitrogen-containing substrates, complex nitrogen-containing substrates, or combinations thereof. The simple nitrogen-containing substrate can include, but is not limited to, ammonium, nitrate, urea, yeast extract, peptones, seed hydrolysates, casein hydrolysates, or combinations thereof. In some embodiments, the complex nitrogen-containing substrate can include, but is not limited to, non-hydrolyzed seed meals, non-hydrolyzed casein, non-hydrolyzed peptides, non-hydrolyzed proteins, non-hydrolyzed amino acids, or combinations thereof.

In the example of FIG. 2, feedstock stream 220 is mixed with the nanobubble-containing liquid carrier stream 210a to form a nanobubble-containing liquid carrier/feedstock stream 210c. In some examples, the feedstock stream 220 is mixed with the liquid carrier stream 210 upstream of the nanobubble generator 230a. In some examples, the feedstock stream 220 can be mixed with both the liquid carrier stream 210 and the nanobubble-containing liquid carrier stream 210b. Thus, fermentation processes described herein encompass generating nanobubbles in liquid carrier mixed with feedstock.

The nanobubble-containing liquid carrier/feedstock stream 210c is sterilized using a sterilization unit 250a, e.g., in a continuous flow sterilization process or a batch sterilization process. Sterilized nanobubble-containing liquid carrier/feedstock stream 210d is flowed into a fermentation tank 270. The sterilized nanobubble-containing liquid carrier/feedstock stream 210d can be added to the fermentation tank 270 in a continuous flow or as a single, discrete injection. Microorganisms are added to fermentation tank 270 via an inoculant stream 260, e.g., before, during, or after addition of the sterilized nanobubble-containing liquid carrier/feedstock stream 210d. Thus, microorganisms are combined with sterilized nanobubble-containing liquid carrier/feedstock in the fermentation tank. Fermentation of the feedstock is performed in the fermentation tank 270, producing fermentation product and fermentation residue.

In some examples, instead of or in addition to sterilization by the sterilization unit 250a upstream of the fermentation tank 270, the nanobubble-containing liquid carrier/feedstock stream can be added to the fermentation tank 270 and sterilized in the fermentation tank 270 using a sterilization unit 250b, e.g., in a batch sterilization process. Microorganisms are added to the fermentation tank 270 via inoculant stream 260 after sterilization is complete.

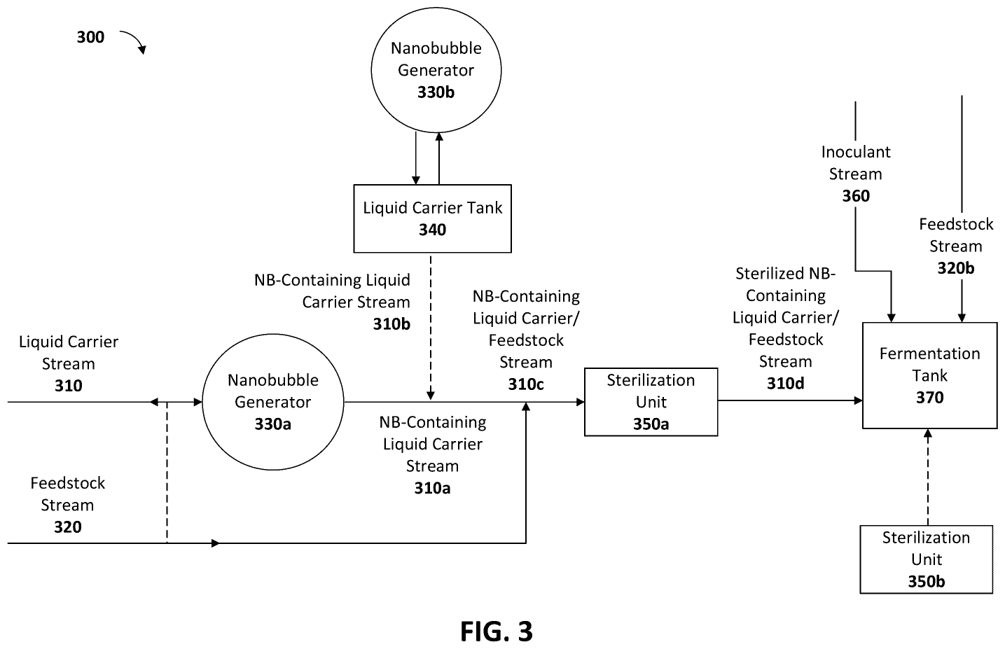

Methods described herein encompass fed-batch fermentation in which feedstock is intermittently added into a fermentation tank. Referring to FIG. 3, an example of a submerged fed-batch fermentation process 300 involving use of nanobubbles is described.

A liquid carrier stream 310 (e.g., a water stream) is flowed into a nanobubble generator 330a where nanobubbles are generated in liquid carrier stream 310 to produce a nanobubble-containing liquid carrier stream 310a. In some implementations, the liquid carrier stream 310 contains only liquid carrier, and in some implementations, the liquid carrier stream 310 contains both liquid carrier and feedstock.

Fermentation processes described herein can include more than one introduction of nanobubble-containing liquid carrier. For example, as shown in FIG. 3, a nanobubble-containing liquid carrier stream 310b from a liquid carrier tank 340 can be mixed with the nanobubble-containing liquid carrier stream 310a. Nanobubbles are generated in liquid carrier from the liquid carrier tank 340 by generating nanobubbles in the liquid carrier along a recirculating flow path using a nanobubble generator 330b.

Feedstock can be mixed with liquid carrier before or after nanobubbles are generated in the liquid carrier. Feedstock can include nutrients and/or media for fermentation of microorganisms. In the example of FIG. 3, feedstock stream 320 is mixed with the nanobubble-containing liquid carrier stream 310a to form a nanobubble-containing liquid carrier/feedstock stream 310c. In some examples, the feedstock stream 320 is mixed with the liquid carrier stream 310 upstream of the nanobubble generator 330a. In some examples, the feedstock stream 320 can be mixed with both the liquid carrier stream 310 and the nanobubble-containing liquid carrier stream 310b. Thus, fermentation processes described herein encompass generating nanobubbles in liquid carrier mixed with feedstock.

The nanobubble-containing liquid carrier/feedstock stream 310c is sterilized in a sterilization unit 350a, e.g., in a continuous flow sterilization process or a batch sterilization process. Sterilized nanobubble-containing liquid carrier/feedstock stream 310d is flowed into a fermentation tank 370. The sterilized nanobubble-containing liquid carrier/feedstock stream 310d can be added to the fermentation tank 370 in a continuous flow or as a single, discrete injection. Microorganisms are added to fermentation tank 370 via an inoculant stream 380, e.g., before, during, or after addition of the sterilized nanobubble-containing liquid carrier/feedstock stream 310d. Thus, microorganisms are combined with sterilized nanobubble-containing liquid carrier/feedstock in the fermentation tank. Feedstock can be intermittently supplied to microorganisms in fermentation tank 370 via a feedstock stream 320b. Fermentation of the feedstock is performed, producing fermentation product and fermentation residue.

In some examples, instead of or in addition to sterilization by the sterilization unit 350a upstream of the fermentation tank 370, the nanobubble-containing liquid carrier/feedstock stream can be added to the fermentation tank 370 and sterilized in the fermentation tank 370 using a sterilization unit 350b, e.g., in a batch sterilization process. Microorganisms are added to the fermentation tank 370 via inoculant stream 380 after sterilization is complete.

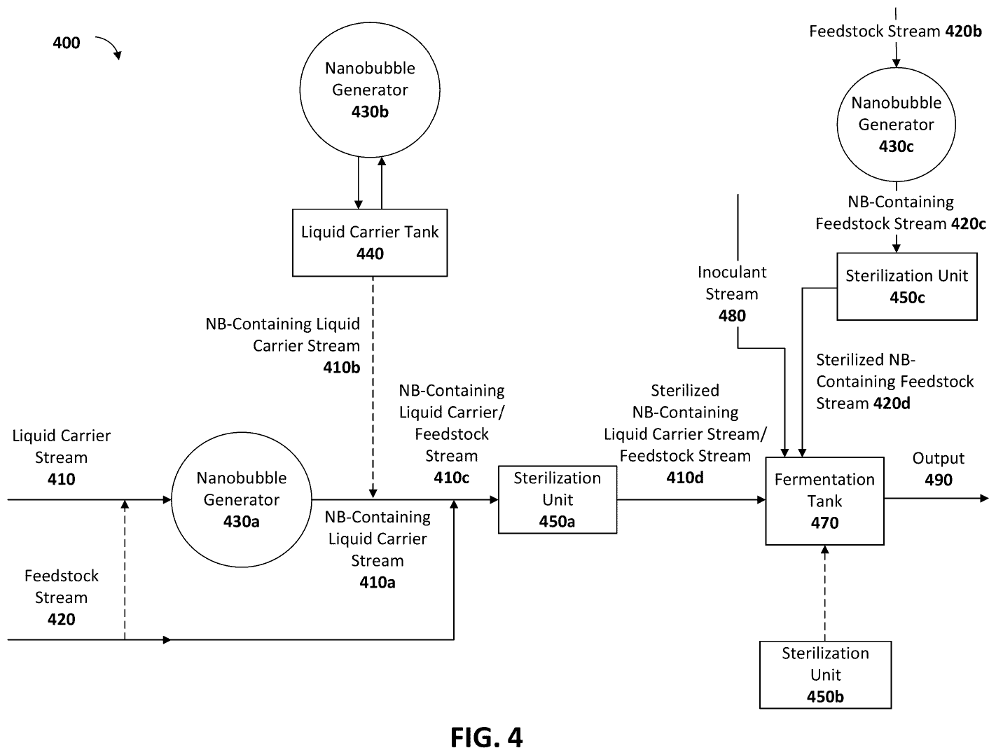

Methods described herein encompass submerged continuous fermentation processes with nanobubbles. Referring to FIG. 4, an example of a submerged continuous fermentation process 400 involving use of nanobubbles is described.

A liquid carrier stream 410 (e.g., a water stream) is flowed into a nanobubble generator 430a where nanobubbles are generated in liquid carrier stream 410 to produce a nanobubble-containing liquid carrier stream 410a. In some implementations, the liquid carrier stream 410 contains only liquid carrier, and in some implementations, the liquid carrier stream 410 contains both liquid carrier and feedstock.

Fermentation processes described herein can include more than one introduction of nanobubble-containing liquid carrier. For example, as shown in FIG. 4, a nanobubble-containing liquid carrier stream 410b from a liquid carrier tank 440 can be mixed with the nanobubble-containing liquid carrier stream 410a. Nanobubbles can be generated in liquid carrier from the liquid carrier tank 440 by generating nanobubbles in liquid carrier along a recirculating flow path using nanobubble generator 430b.

Feedstock can be mixed with liquid carrier before or after nanobubbles are generated in the liquid carrier. Feedstock can include nutrients and/or media for fermentation of microorganisms. In the example of FIG. 4, feedstock stream 420 is mixed with the nanobubble-containing liquid carrier stream 410a to form a nanobubble-containing liquid carrier/feedstock stream 410c. In some examples, the feedstock stream 420 is mixed with the liquid carrier stream 410 upstream of the nanobubble generator 430a. In some examples, the feedstock stream 420 can be mixed with both the liquid carrier stream 410 and the nanobubble-containing liquid carrier stream 410b. Thus, fermentation processes described herein encompass generating nanobubbles in liquid carrier mixed with feedstock.

The nanobubble-containing liquid carrier/feedstock stream 410c is sterilized in a sterilization unit 450a, e.g., in a continuous flow sterilization process or a batch sterilization process. Sterilized nanobubble-containing liquid carrier/feedstock stream 410d is flowed into a fermentation tank 470. The sterilized nanobubble-containing liquid carrier/feedstock stream 410d can be added to the fermentation tank 470 in a continuous flow or as a single, discrete injection. Microorganisms are added to fermentation tank 470 via an inoculant stream 480, e.g., before, during, or after addition of the sterilized nanobubble-containing liquid carrier/feedstock stream 410d. Thus, microorganisms are combined with sterilized nanobubble-containing liquid carrier/feedstock in the fermentation tank.

Additional feedstock can be continuously supplied to fermentation tank 470 via a sterilized nanobubble-containing feedstock stream 420d. To produce sterilized nanobubble-containing feedstock stream 420d, nanobubbles are generated in a feedstock stream 420b by a nanobubble generator 430c to produce nanobubble-containing feedstock stream 420c, which is sterilized using a sterilization unit 450c, e.g., in a continuous sterilization process, to produce sterilized nanobubble-containing feedstock stream 420d. Fermentation of the feedstock is performed, producing fermentation product and fermentation residue.

In some examples, instead of or in addition to sterilization by the sterilization unit 450a upstream of the fermentation tank 470, the nanobubble-containing liquid carrier/feedstock stream can be added to the fermentation tank 470 and sterilized in the fermentation tank 470 using a sterilization unit 450b, e.g., in a batch sterilization process. Microorganisms are added to the fermentation tank 470 via inoculant stream 480 after sterilization is complete.

A continuous flow of overflow fermentation product and cells can be output from fermentation tank 470 in output 490.

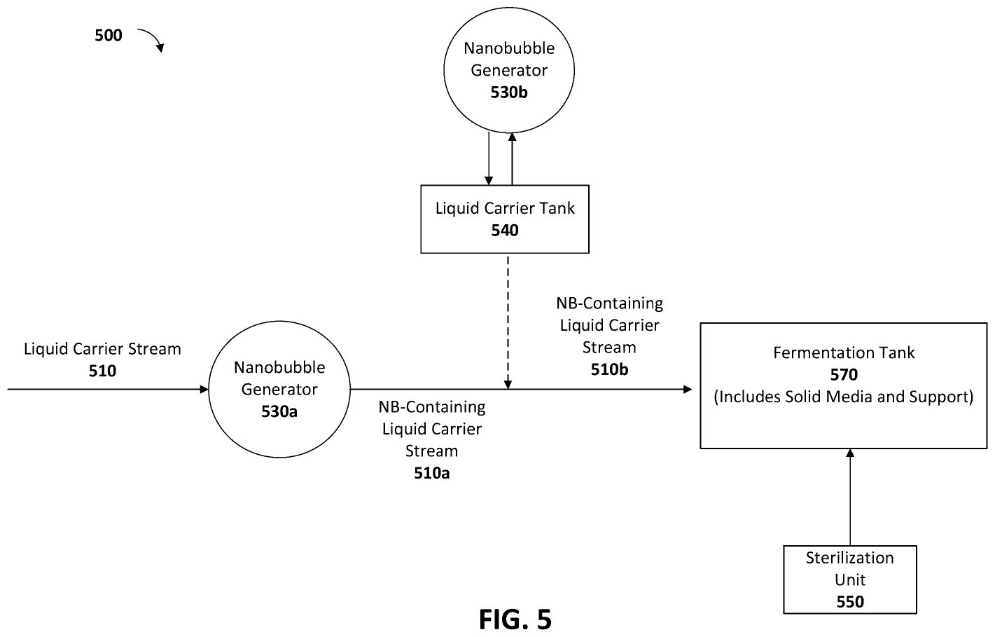

Fermentation processes using nanobubbles described herein encompass liquid state fermentation processes and solid state fermentation processes. Referring to FIG. 5, an example of a solid state fermentation process involving use of nanobubbles is described.

A nanobubble generator 530a is used to generate nanobubbles in a liquid carrier stream 510 (e.g., a water stream), producing a nanobubble-containing liquid carrier stream 510a. Additional nanobubble-containing liquid carrier can be added to fermentation process 500 by mixing nanobubble-containing liquid carrier stream 510b and nanobubble-containing liquid carrier stream 510a. Nanobubble-containing liquid carrier stream 510b is generated in liquid carrier from a liquid carrier tank 540 by generating nanobubbles in the liquid carrier along a recirculating flow path using a nanobubble generator 530b.

Nanobubble-containing liquid carrier stream 510a, optionally combined with nanobubble-containing liquid carrier stream 510b, is flowed into a fermentation tank 570, which includes solid feedstock and support that allows fermentation to take place in the tank. The contents of the fermentation tank 570, including nanobubble-containing liquid carrier stream 510a, are sterilized in the fermentation tank 570 using a sterilization unit 550, e.g., in a batch sterilization process. Microorganisms are added to the fermentation tank 570 after sterilization is complete.

Non-limiting examples of sterilization processes include moist heat (e.g., steam) processes, dry heat processes, irradiation processes (e.g., ultraviolet, gamma, electron beam, or plasma irradiation processes), chemical processes (e.g., ethylene oxide vapor, vaporized hydrogen peroxide, chlorine dioxide gas, vaporized peracetic acid, and nitrogen dioxide), electromagnetic processes (e.g., pulsed electromagnetic fields), sonication processes, and a combination of any of these.

Non-limiting examples of a sterilization unit include an autoclave, a clean-in-place (CIP) system, a sterilization-in-place (SIP) system, a dry heat sterilizer, an oven, a steam sterilizer, a UV chamber, a sonicator, and a combination of any of these.

A nanobubble composition comprising nanobubbles in a liquid carrier can be introduced in a discontinuous manner or a continuous manner in fermentation processes described herein. In some embodiments, fermentation processes described herein include one or more (e.g., 1, 2, 3, 4, 5 or more) introductions of a nanobubble composition comprising nanobubbles in a liquid carrier. In some embodiments, fermentation processes described herein include a single introduction of a nanobubble composition comprising nanobubbles in a liquid carrier. In some embodiments, fermentation processes described herein include a continuous introduction of a nanobubble composition comprising nanobubbles in a liquid carrier.

Any suitable microorganism can be used in fermentation processes described herein. Non-limiting examples of microorganisms include bacteria (e.g., E. coli), fungi (e.g., yeast), algae, viruses, and a combination of any of these.

Any suitable liquid carrier can be used in fermentation processes described herein. In some embodiments, the liquid carrier comprises an aqueous carrier. Non-limiting examples of an liquid carrier include water, saline, buffers, aqueous solutions, and a combination of any of these.

### EXAMPLES

The following examples described in this application are offered to illustrate the methods provided herein and are not to be construed in any way as limiting.

### Example 1: Stability of Nanobubbles During Sterilization

This Example describes the stability of nanobubbles in a liquid carrier during sterilization, which was performed by incubating a sample of nanobubbles in the liquid carrier at 121° C. for 30 minutes. The concentration of nanobubbles in each sample was measured before and after sterilization. The experiment was performed with two samples having different starting concentrations of nanobubbles: 45 million/mL and 150 million/mL. As shown in Table 1 below, sterilization did not significantly alter the concentration of nanobubbles in each sample. These results demonstrate that nanobubbles can survive the sterilization processes used during fermentation.

TABLE 1
Stability of nanobubbles during sterilization

Nanobubble Concentration
(million/mL)

| Sample | Sample 1 | Sample 2 |
|---|---|---|
| Nanobubble concentration | 45 | 150 |
| before sterilization | | |
| Nanobubble concentration | 44 | 148 |
| after sterilization | | |

### Example 2: Effect of Nanobubbles on Fermentation

This Example describes the effect of nanobubbles in a liquid carrier on sugar fermentation by yeast, which produces ethanol and carbon dioxide. Yeast cultures were prepared with and without nanobubbles and incubated at 35° C. for up to 48 hours. Fermentation rate was assessed by measuring the production of ethanol at the indicated time points. As shown in Table 2 below, the yeast culture with nanobubbles produced significantly more ethanol at each time point compared to the yeast culture without nanobubbles. These results demonstrate that nanobubbles can improve the fermentation rate.

TABLE 2
Effect of sterilized nanobubble on yeast fermentation of sucrose

| | Alcohol content (% v/v) | | Rate enhancement |
|---|---|---|---|
| Fermentation time | Without | With sterilized | due to nanobubbles |
| (hrs) | nanobubbles | nanobubbles | (%) |
| 15 | 2.08 | 3.23 | 56 |
| 20 | 3.24 | 4.73 | 46 |
| 24 | 4.74 | 6.11 | 29 |
| 40 | 7.86 | 10.04 | 28 |
| 45 | 9.48 | 11.65 | 23 |
| 48 | 10.17 | 12.23 | 20 |

### Other Embodiments

It is to be understood that while the document has been described in conjunction with the detailed description thereof, the foregoing description is intended to illustrate and not limit the scope of the document. Other aspects, advantages, and modifications are within the scope of the following claims. For example, fermentation methods described herein encompass filtration of feedstock and/or microorganisms to reduce contaminants.

What is claimed is:

1. A fermentation process comprising:
(a) providing, in a fermentation tank, a fermentable composition comprising (i) a sterilized nanobubble composition comprising nanobubbles in a liquid carrier, (ii) a feedstock, and (iii) a microorganism;
(b) fermenting the fermentable composition to produce a fermentation product and fermentation residue; and
(c) separating the fermentation product from the fermentation residue.

2. The fermentation process of claim 1, comprising:
(a) adding (i) a nanobubble composition comprising nanobubbles in a liquid carrier and (ii) feedstock to the fermentation tank;
(b) sterilizing the contents of the tank; and
(c) adding the microorganism to the tank.

3. The fermentation process of claim 1, comprising:
(a) combining a sterilized composition comprising nanobubbles in a liquid carrier with a feedstock to form a precursor composition;
(b) adding the precursor composition to the fermentation tank; and
(c) adding the microorganism to the tank.

4. The fermentation process of claim 1, comprising:
(a) combining a sterilized composition comprising nanobubbles in a liquid carrier with a feedstock to form a precursor composition; and
(b) combining the precursor composition with a microorganism present in the fermentation tank.

5. The fermentation process of claim 1, comprising: (a) forming a sterilized precursor composition comprising nanobubbles in a liquid carrier and a feedstock; (b) adding the sterilized precursor composition to the fermentation tank; and (c) adding the microorganism to the tank.

6. The fermentation process of claim 1, comprising: (a) forming a sterilized precursor composition comprising nanobubbles in a liquid carrier and a feedstock; and (b) combining the sterilized precursor composition with a microorganism present in the fermentation tank.

7. The fermentation process of claim 5, wherein forming the sterilized precursor composition comprises combining a sterilized nanobubble composition comprising nanobubbles in a liquid carrier with a sterilized feedstock to form the sterilized precursor composition.

8. The fermentation process of claim 5, wherein forming the sterilized precursor composition comprises:
(a) combining a nanobubble composition comprising nanobubbles in a liquid carrier with a feedstock to form a sterilizable composition; and
(b) sterilizing the sterilizable composition to form the sterilized precursor composition.

9. The fermentation process of claim 3, comprising forming the precursor composition upstream of and in-line with the fermentation tank.

10. The fermentation process of claim 5, comprising forming the sterilized precursor composition upstream of and in-line with the fermentation tank.

11. The fermentation process of claim 1, wherein the concentration of nanobubbles in the sterilized nanobubble composition comprising nanobubbles in a liquid carrier is at least 10⁶ nanobubbles per cm³, at least 10⁷ nanobubbles per cm³, at least 10⁸ nanobubbles per cm³, at least 10⁹ nanobubbles per cm³, at least 10¹⁰ nanobubbles per cm³, or at least 10¹¹ nanobubbles per cm³.

12. The fermentation process of claim 1, comprising conducting the fermentation process using a continuous introduction of the nanobubble composition comprising nanobubbles in a liquid carrier.

13. The fermentation process of claim 1, comprising conducting the fermentation process using a single introduction of the nanobubble composition comprising nanobubbles in a liquid carrier.

14. The fermentation process of claim 1, wherein the liquid carrier comprises an aqueous carrier.

15. The fermentation process of claim 1, wherein the microorganism is selected from the group consisting of bacteria, fungi, yeast, and combinations thereof.
